(19) United States
(12) Patent Application Publication
(10) Pub. No.: US 2022/0031612 A1
(43) Pub. Date: Feb. 3, 2022

(54) URICASE GEL PREPARATION FOR EXTERNAL USE, PREPARATION METHOD THEREFOR AND USE THEREOF

(71) Applicant: CHINA PHARMACEUTICAL UNIVERSITY, Nanjing (CN)

(72) Inventors: Jianhua CHEN, Nanjing (CN); Xiuhua HUANG, Nanjing (CN); Yuxia WANG, Nanjing (CN)

(21) Appl. No.: 17/275,958

(22) PCT Filed: Sep. 2, 2019
(86) PCT No.: PCT/CN2019/103968

§ 371 Date: Mar. 12, 2021

(30) Foreign Application Priority Data
Sep. 13, 2018 (CN) 201811065580.7

(51) Int. Cl. A61K 9/06; A61K 47/38; A61K 9/00; A61K 38/44

(52) U.S. Cl. CPC A61K 9/06; A61K 47/38; C12Y 107/03003; A61K 38/44; A61K 9/0014

(57) ABSTRACT

A uricase gel preparation for external use comprises the following components by weight: greater than 0 and less than or equal to 1.0% of uricase, 0.05-20% of an aqueous gel matrix, 0.15-20% of a protein protectant, 2-25% of a humectant, 0.01-0.20% of a chelating agent, 0.001-3.0% of a transdermal enhancer, 0.005-0.5% of a preservative and the balance of water for injection. The pH of the preparation is adjusted to 5-10 by a pH adjuster. The method for preparing the gel preparation comprises: preparing a standby substance from some of the components; respectively formulating each of the remaining components with a water for injection; adding the preservative into the standby substance and adjusting the pH; adding the protein protectant, uricase and water for injection; stirring uniformly same and then subpackaging same to obtain the gel preparation.

### TECHNICAL FIELD

The invention relates to a gel preparation for external use of uricase, which can be used as a pharmaceutical preparation for the treatment of hyperuricemia, gout and diseases caused by hyperuricemia, and also relates to a preparation method and application of the gel preparation, belonging to biological medicine preparation technology field.

### BACKGROUND TECHNIQUE

According to the inventor, as the end product of human purine metabolism, uric acid (UA) is mainly produced by the metabolism of the liver, gastrointestinal tract, etc., enters the blood and forms a uric acid metabolism pool. When the metabolism of uric acid in the human body is impaired, the hyperuricemia is induced by overproduction and renal underexcretion of uric acid. The diagnostic index of hyperuricemia is usually that women's serum uric acid level is greater than 6 mg/dL [360 μM (M means mol/L, the same below)], and men's serum uric acid level is greater than 7 mg/dL (420 μM). Long-term supersaturated uric acid forms sodium urate crystals and deposits in the joint cavity or soft tissues to form tophi. The main clinical features of gout are hyperuricemia, and repeated acute and chronic attacks of gout caused by hyperuricemia, inflammation of the joints, joint deformities, uric acid urinary tract stones, causing damage to the kidneys, causing interstitial nephritis, renal failure and so on. In addition, serum and/or intracellular urate can stimulate the renin-angiotensin-aldosterone system and induce hypertension. Studies have also found that uric acid is involved in diabetic nephropathy, calcineurin inhibitor nephrotoxicity and acute kidney injury (Johnson R J, Bakris G L, Borghi C, et al. American Journal of Kidney Diseases. 2018; 71(6): 851-865). Gout is often accompanied by clinical manifestations such as abdominal obesity, hyperlipidemia, hypertension, type 2 diabetes and cardiovascular disease.

Serum uric acid is mainly excreted from the body by the kidneys, intestines, and skin sweat glands. The kidney is the main organ for uric acid excretion. Uric acid can freely pass through the glomerulus and is reabsorbed and secreted in the proximal tubules. Most of the secreted uric acid is reabsorbed, and less than 10% is excreted. The process of reabsorption and secretion relies on ion channels and related urate transporters.

The excretion of uric acid in the intestines accounts for about one-third of the daily excretion. The transport mechanism of uric acid in the intestine is not clear, but GLUT9 and ABCG2 transport uric acid to the intestine. The knockout of GLUT9 gene in the intestine can cause hyperuricemia, and the knockout of ABCG2 gene can cause hyperuricemia and “overload” hyperuricosuria (Johnson R J, Bakris G L, Borghi C, et al. American Journal of Kidney Diseases. 2018; 71(6): 851-865).

Human skin contains about 2-5 million sweat glands, which secrete sweat to the surface of the skin under the control of sympathetic nerves (autonomic nerves) to avoid excessive temperature of the body caused by the rise of the ambient temperature and subsequent body dysfunction, Provide a homeostasis for the balance of the human body and the environment. The skin is the most important excretory organ besides the lungs, kidneys, and intestines. Through the sweating effect of sweat glands, uric acid, urea, etc. can be excreted out of the body, and it can assist or replace the partial function of kidneys. It can be regarded as a special form Kidneys (Hanafusa N, Lodebo B T, Shah A, et al. Journal of Renal Nutrition. 2017; 27 (5): 295-302). The excretion of sweat glands and kidneys is very closely coordinated. When the excretion of the kidneys is impaired, the content of nitrogen metabolites such as urea in sweat increases, which can compensate partially for the lack of kidney function. Studies have shown that uric acid in sweat is about 24.5 μM, which accounts for about 6.3% of serum uric acid (SUA) level (Huang C T, Chen M L, Huang L L, et al. Chinese Journal of Physiology. 2002; 45 (3):109-115). Uric acid passes through the skin barrier through sweat glands to reach the surface of the skin, providing the theoretical basis for the development of topical drug formulations that degrade uric acid.

Gout is a “disease of wealth”. With the improvement of living standards, the prevalence of hyperuricemia and gout in the Chinese population is gradually increasing. According to Meta-Analysi s, in Mainland China, the prevalence rate of gout in 2014 was 1.4%, while the prevalence rate of gout from 2000 to 2005 was only 0.9% (Liu R, Han C, Wu D, et al. Biomed Research International. 2015; (15, supplement):1-12). Epidemiological studies have found that starting from the first gout patients in China in 1948, prevalence rate of hyperuricemia patients was 10% in 2016, with the number of patients reaching 135 million, and about 17 million patients with gout. Gout has become the second largest metabolic disease in Mainland China, and the third type of affluence disease after hyperglycemia and hyperlipidemia.

Gout treatment is a long-term process. In addition to reasonable weight loss, avoid alcohol (especially beer and spirits), sugary drinks, overeating, and excessive intake of meat and seafood, and encourage low-fat dairy products and regular exercise. Colchicine is the first-line treatment for acute gout attacks and other drugs such as non-steroidal anti-inflammatory drugs, glucocorticoids and allopurinol (Benn C L, Dua P, Gurrell R, et al. Frontiers in medicine. 2018; 5:160-188). However, for patients with gout who are confirmed to have urate crystals, severe chronic tophi even if the maximum dose of urate-lowering treatment drugs (including combination drugs) are applied, target serum uric acid levels fail to achieve less than 6 mg/dl. Uricase can be recommended as the drugs for treatment (Richette P, Doherty M, Pascual E, et al. Annals of the Rheumatic Diseases. 2017; 76 (1):29-42). In addition, in 2016, the European League Against Rheumatism (EULAR) recommended that patients receiving urate-lowering treatment should monitor their serum uric acid levels and allow them below 360 μM; for patients with severe gout (tophi, chronic arthropathy, frequent attacks), the serum uric acid level should be less than 300 μM to promote the faster dissolution of uric acid crystals until all uric acid crystals are dissolved and the gout disappears. During the long-term treatment, the uric acid level should not be lower than 180 μM.

It was found that three uricase-based drugs have been developed internationally, namely: (1) Uricozyme from Aspergillus flavus approved in France and Italy in the 1970s; (2) Uricozyme in the United States in 2000 Recombinant uricase (Rasburicase) expressed by yeast genetically engineered bacteria with the uricase gene (derived from Aspergillus flavus) approved by the European Union for marketing; (3) Pegloticase, polyethylene glycol-modified recombinant uricase gene from pigs and baboons, approved by the US FDA in 2010. The first two uricase drugs are derived from microorganisms and have strong immunogenicity. Pegloticase is expressed by the chimeric uricase gene from two mammals, pigs and baboons. After chemical modification of polyethylene glycol, the half-life of intravenous administration of Pegloticase was significantly increased. However, 41% of patients have developed high titers against Pegloticase Antibodies, among which 40% of patients have developed high-titer antibodies against PEG (Lipsky P E, Calabrese L H, Kavanaugh A, et al. Arthritis Research & Therapy. 2014; 16(2):(R60)18). Clinical studies have found that 45% of patients have infusion reactions after intravenous infusion, including chest tightness (15%), flushing (12%), and dyspnea (11%) etc. (Baraf H S, Yood R A, Ottery F D, et al. Journal of Clinical Rheumatology. 2014; 20(8):427-432). The above-mentioned uricase drugs are all freeze-dried powder injections, which are applied to the human body by intravenous drip. Although the effect of degrading uric acid is rapid, it can cause acute attacks of gout in 77% of patients (Lyseng Williamson K A. Drugs. 2011; 71(16):2179-2192). In the process of drug development, the low immunogenicity of mammalian uricase and the high specific activity of microbial uricase have made the development and application of recombinant uricase from these two sources as both directions.

There is also a nanoparticle combination treatment method that degrades urate crystals, using double emulsification solvent evaporation method to evaporate PLGA emulsification solvent to prepare nanoparticles containing uricase and diclofenac. Due to the small particle size and large specific surface area of nanoparticles, compared with other drug delivery systems, the permeability of the drug through the skin is improved. On this basis, the further prepared nanoparticle gel delivers drugs to the synovial fluid through the skin to play a therapeutic effect. The preparation process of the dosage form is complicated and the stability is poor. The half-life of the nanoparticle gel agent is only 45.22 days under refrigerated conditions, and it is difficult for the macromolecular drug to penetrate the skin barrier and exert its efficacy (Tiwari S, Dwivedi H, Kymonil K M, et al. Drug Deliv Transl Res. 2015; 5(3):219-230).

The inventor's research group is committed to research uricase and its pharmaceutical preparations, and has applied for several Chinese invention patents, such as the Chinese invention patent with patent number CN2014100480719, authorized announcement number CN103834623B; application number CN2015100667452, application publication number Chinese invention patent application for CN104630168A; application number CN2016103167091, Chinese invention patent application for application publication number CN105838686A. At present, the inventor's research group has made further research results in uricase preparations.

### SUMMARY OF THE INVENTION

The main purpose of the present invention is to overcome the problems existing in the prior art and provide a gel preparation for external use of uricase, which is applied on the skin surface. The preparation is easy to prepare, is convenient to use, has good stability and has a stable effect. It is easy to control the serum uric acid levels autonomously during medication, and the efficacy of the preparation does not depend on the entry of uricase into the body through the skin barrier. The transdermal excretion of urate has been induced by uricase on skin surface no which uricase disintegrate urate into allantoin. In addition, the present invention also provides the preparation method of the preparation and the uses of the preparation.

The technical solutions of the present invention to solve its technical problems are as follows:

A gel preparation for external use of uricase, which is characterized in that: the preparation is composed of the following components in weight percentage:

Main medicine: Uricase Greater than 0 and less than or equal to 1.0%,

Aqueous gel matrix 0.05-20%,

Protein protector 0.15-20%,

Moisturizer 2-25%,

Chelating agent 0.01-0.20%,

Penetration enhancer 0.001-3.0%,

Preservative 0.005-0.5%,

The balance is water for injection;

The pH of the formulation is adjusted to 5-10 by a pH adjuster.

Preferably, the uricases include natural uricases, recombinant uricases, chimeric uricases, and fusion uricases. The natural uricases are derived from prokaryotes or eukaryotes. The recombinant uricases, chimeric uricases, fusion uricases are prepared by bioengineering technology. The natural uricases include uricases derived from microorganisms and uricases derived from mammals, the microorganisms include Candida utilis and Aspergillus flavus, and the mammals include pigs and dogs. The recombinant uricases include recombinant uricases expressed by natural uricase genes prepared by bioengineering technology. The chimeric uricases include a uricase chimeric protein that is chimeric and recombinantly expressed by nucleotide sequences between different natural uricase genes. The fusion uricase includes human serum albumin-uricase fusion protein (from natural uricase gene and human serum albumin gene fusion, recombinant expression) and Fc-uricase fusion protein (from natural uricase gene and humanized antibody Fc fragment nucleotide sequence fusion, recombinant expression).

More preferably, the uricase has a chemical modification, and the chemical modification includes PEG modification. Preferably, the aqueous gel matrix is selected from natural polymer, semi-synthetic polymer or synthetic polymer; said natural polymers include starch, alginate, gum arabic, tragacanth, agar and gelatin; Semi-synthetic polymers include modified cellulose and modified starch. The modified cellulose includes carboxymethyl cellulose and methyl cellulose; and the synthetic polymers include carbomer and sodium polyacrylate.

More preferably, the aqueous gel matrix is Carbomer 934, Carbomer 940, Carbomer 941, Carbomer 942, Carbomer 971, Carbomer 974, Carbomer 980, Carbomer 981. One or a combination.

Preferably, the protein protective agents are selected from one or a combination of bovine serum albumin (BSA), mannitol, sucrose, sodium citrate, and sorbitol.

The moisturizers are selected from one or a combination of glycerin, propylene glycol, ethanol, and hyaluronic acid.

The chelating agents are selected from one or a combination of salts of ethylenediaminetetraacetic acid, and the salts of ethylenediaminetetraacetic acid include disodium ethylenediaminetetraacetic acid (EDTA-2Na), ethylenediaminetetraacetic acid dipotassium salt (EDTA-2K).

The penetration enhancers are selected from one or a combination of azone, propylene glycol, hyaluronic acid, cholate, deoxycholate, urea, cyclodextrin, Tween-80.

The preservatives are selected from one or a combination of benzoic acid, sodium benzoate, methyl p-hydroxybenzoate, ethyl p-hydroxybenzoate, phenol, and sorbic acid.

The pH adjusting agents are selected from one of triethanolamine, NaOH, and NaHCO₃.

Preferably, the content of the uricase is 0.001-0.1%; the pH of the preparation is 6-9.

The present invention also provides: a preparation method of the aforementioned uricase gel preparation for external use, which is characterized by including the following steps:

The first step is to take an appropriate amount of water for injection, add transdermal enhancer, moisturizer, and chelating agent, and dissolve and stir evenly; then add the aqueous gel matrix, swell and/or dissolve and then stir evenly, and sterilize with moist heat.

The second step is to add the protein protectant, preservative, and main drug uricase to dissolve it in water for injection, and filter it through a microporous membrane for sterilization respectively.

The third step, the preservative obtained in the second step is added to the reserve obtained in the first step, and the pH value is adjusted to 5-10 with a pH adjuster, and then the remaining protein protectant and the main drug uricase in the second step are added. Add the remainder of water for injection, stir evenly, and divide into small packages to obtain the finished product of uricase gel preparation for external use.

Preferably, in the first step, the swelling time of aqueous gel matrix in water for injection is overnight, and the humid heat sterilization conditions are 115° C. for 30 minutes. In the second step, the pore size of the microporous membrane is 0.22 μm. In the third step, the adjusted pH is 6-9. When sub-packing, sub-packing into a pharmaceutically acceptable container, the container includes pharmaceutical aluminum tube, plastic tube, aluminum-plastic tube, polyethylene composite tube, or when sub-packing, coating a gel patch made with hygienic materials; after packaging, store the content in a refrigerator at 4° C.

The present invention also provides: the use of the aforementioned uricase gel preparation for external use in the preparation of a medicine or a pharmaceutical composition, the effect of which is to reduce uric acid, or treat hyperuricemia, or treat gout, or treat diseases caused by hyperuricemia.

Compared with the prior art, the beneficial effects of the present invention are as follows:

The medication route of the gel preparation of the present invention is to apply externally to the skin surface to degrade the uric acid secreted by the sweat glands on the skin surface to generate allantoin, thereby forming a concentration gradient of uric acid inside and outside the skin, and promoting the sweat glands to continuously secrete uric acid and other metabolism in the form of microflow. The product exerts a significant effect of lowering uric acid level in the body. The efficacy of this preparation does not depend on the entry of uricase into the body through the skin barrier, unlike freeze-dried powder injections that need to be administered intravenously into the body, avoiding allergic reactions and infusion reactions of uricase and antibody production. The preparation has the effect of degrading uric acid. In addition to being used to treat hyperuricemia and gout, it can also be used to treat complications caused by increased serum uric acid concentration, such as diabetes, hypertension, interstitial nephritis, renal failure and other cardiovascular diseases and other adjuvant treatments.

The preparation method of the present invention can not only maintain the activity of uricase enzyme but also keep the stability of the uricase gel preparation for a long time. The preparation process of the invention is simple. The preparation stability is good. The use is convenient. The medicinal effect is stable and the serum uric acid level is easily controlled independently during the medication process.

### DESCRIPTION OF THE DRAWINGS

FIG. 1 is a standard curve diagram of uric acid in Example 9 of the present invention.

FIG. 2 is a standard curve diagram of uric acid determined by high performance liquid chromatography in Example 11 of the present invention.

FIG. 3 is an exemplary diagram of detecting uric acid solution by high performance liquid chromatography in Example 11 of the present invention.

FIG. 4 is a graph showing the results of determination of uric acid content on the skin surface of each experimental group in Example 11 of the present invention.

FIG. 5 is a graph showing the results of determination of uric acid content in skin tissues of each experimental group in Example 11 of the present invention.

FIG. 6 is a standard curve diagram of allantoin measured by high performance liquid chromatography in Example 12 of the present invention.

FIG. 7 is a graph showing the results of determination of allantoin content on the skin surface of each experimental group in Example 12 of the present invention.

FIG. 8 is a schematic diagram of the results of Example 13 of the present invention.

### DETAILED DESCRIPTION

Hereinafter, the present invention will be further described in detail with reference to the accompanying drawings and the embodiments. However, the present invention is not limited to the Examples given. The test methods in the following Examples are conventional methods unless otherwise specified; reagents and materials, unless otherwise specified, can be obtained through commercial channels.

### Example 1. Preparation Scheme 1 of Uricase Gel Preparation for External Use

The uricase gel preparation for external use in this embodiment consists of the following components (calculated as 100 g gel):

Recombinant human-pig chimeric uricase (main drug) 5 mg (0.005%)

Carboxymethyl cellulose (aqueous gel matrix) 0.20 g (0.2%)

BSA (protein protectant) 0.2 g (0.2%)

Glycerin (humectant) 5 g (5%)

EDTA-2K (chelating agent) 50 mg (0.05%)

Hyaluronic acid (transdermal enhancer) 12 mg (0.012%)

Sodium benzoate (preservative) 0.3 g (0.3%)

The balance is water for injection.

Use 15% triethanolamine solution (pH adjuster) to adjust the pH of the formulation to 7.4.

The preparation method of the uricase gel preparation for external use in this embodiment includes:

(1) Preparation of the main drug recombinant human porcine uricase: according to Chinese patent: 104630168A, construct recombinant E. coli engineering bacteria, inoculate the engineering bacteria on LB solid medium, cultivate at 37° C. for activation, and pick a single colony in LB after 12 hours. In the liquid medium, continue to incubate at 37° C. for 12 h, and then add the fermentation medium at a volume ratio of 1% inoculum, 200 rpm, 37° C. for 4 h, add IPTG to induce the expression of uricase protein, continue the culture for 6 h and collect the wet bacteria by centrifugation. Add Tris-Hcl buffer to resuspend the pellet, and ultrasonically break the cell wall. The conditions for breaking the wall are: 60% power, 3 s over 3 s, 30 min. After centrifugation of the broken wall bacteria solution, the precipitate is collected, and the precipitate is resuspended by adding Tris-Hcl buffer. After centrifugation, the supernatant is collected, and the target protein is salted out using ammonium sulfate fractionated precipitation, and then the precipitate is washed with distilled water 3 times. Add Tris-Hcl buffer to reconstitute, and obtain the crude enzyme solution of human pig chimeric uricase.

(2) Add hyaluronic acid, glycerin, EDTA-2K into 60% of total weight of water for injection, dissolve them and stir evenly; then add carboxymethyl cellulose, stir evenly after dissolution, and sterilize with moist heat at 115° C. for 30 min to get a spare;

(3) Add sodium benzoate, BSA, and the main drug recombinant human-porcine chimeric uricase respectively to an appropriate amount of water for injection to dissolve, and filter it through a 0.22 μm microporous membrane for sterile filtration.

(4) Add the sodium benzoate in (3) to the spare of (2), adjust the pH to 7.4 with 15% triethanolamine solution, and then combine the BSA in (3), the main drug recombinant human-pig chimeric uricase and the remaining of water for injection added to it. Stir it evenly and dispense it into sterile aluminum tubes to obtain the finished product of uricase gel preparation for external use, and store it in a refrigerator at 4° C. for later use.

### Example 2. Preparation Scheme 2 of Uricase Gel Preparation for External Use

The uricase gel preparation for external use of this embodiment consists of the following components (calculated as 100 g gel):

Candida utilis uricase (main drug) 20 mg (0.02%) (purchased from sigma company)

Sodium alginate (aqueous gel matrix) 0.5 g (0.5%)

BSA (protein protectant) 0.15 g (0.15%)

Glycerin (humectant) 10 g (10%)

EDTA-2Na (chelating agent) 40 mg (0.04%)

Hyaluronic acid (transdermal enhancer) 15 mg (0.015%)

Sorbic acid (preservative) 50 mg (0.05%)

The balance is water for injection.

Use 15% NaOH solution (pH adjuster) to adjust the pH of the formulation to 7.5.

The preparation method of the uricase gel preparation for external use in this embodiment includes:

(1) Take 60% by weight of the total water for injection, add hyaluronic acid, glycerin, EDTA-2Na and dissolve and stir evenly; then add sodium alginate, after night swelling and dissolution, sterilize at 115° C. for 30 minutes with humid heat Spare;

(2) Add sorbic acid, BSA, and the main drug Candida utilis uricase to dissolve them in water for injection, and filter them through a 0.22 μm microporous membrane for sterilization.

(3) Add the sorbic acid in (2) to the reserve of (1), and adjust the pH to 7.5 with 15% NaOH solution, then add BSA, the main drug Candida utilis uricase in (2) and the remaining water for injection. Stir it evenly and dispense it into sterile polyethylene composite tubes to obtain the finished product of uricase gel preparation for external use, and store it in a refrigerator at 4° C. for later use.

### Example 3: Preparation Scheme 3 of Uricase Gel Preparation for External Use

The uricase gel preparation for external use in this embodiment consists of the following components (calculated as 100 g gel):

Uricase from Aspergillus flavus (main drug) 30 mg (0.03%) (purchased from Shanghai Guyan Biological Company)

Carbomer 941 (aqueous gel base) 0.25 g (0.25%)

Mannitol (protein protective agent) 18 g (18%)

Glycerin (humectant) 10 g (10%)

EDTA-2K (chelating agent) 60 mg (0.06%)

Propylene glycol (dermal penetration enhancer) 1.5 g (1.5%)

Methyl p-hydroxybenzoate (preservative) 10 mg (0.01%)

The balance is water for injection.

Use 15% NaOH solution (pH adjuster) to adjust the pH of the formulation to 8.5.

The preparation method of the uricase gel preparation for external use in this embodiment includes:

(1) Take 60% of the total weight of water for injection, add glycerin, propylene glycol, EDTA-2K, and dissolve them and stir evenly; then add carbomer 941, after swelling and dissolving for night, sterilize with moist heat at 115° C. for 30 minutes for use thing;

(2) Add methylparaben, mannitol, and the main drug Aspergillus flavus-derived uricase to dissolve them in water for injection, and filter them through a 0.22 μm microporous membrane to sterilize them for later use;

(3) Add the methyl parahydroxybenzoate in (2) to the reserve of (1), adjust the pH to 8.5 with 15% NaOH solution, and then combine the mannitol in (2) with the main drug Aspergillus flavus-derived uricase and add the remaining water for injection. Stir evenly, and dispense into medical aluminum-plastic tubes to obtain the finished product of uricase gel preparation for external use, and store it in a refrigerator at 4° C. for later use.

### Example 4. Preparation Scheme 4 of Uricase Gel Preparation for External Use

The uricase gel preparation for external use in this embodiment consists of the following components (calculated as 100 g gel):

Polyethylene glycol modified uricase (main drug) 45 mg (0.045%)

Methyl cellulose (aqueous gel matrix) 0.3 g (0.3%)

Mannitol (protein protective agent) 18 g (18%)

Glycerin (humectant) 15 g (15%)

EDTA-2Na (chelating agent) 50 mg (0.05%)

Propylene glycol (dermal penetration enhancer) 2.5 g (2.5%)

Benzoic acid (preservative) 10 mg (0.01%)

The balance is water for injection.

Use 10% NaHCO₃ solution (pH adjuster) to adjust the pH of the formulation to 8.0.

The preparation method of the gel preparation for external use of uricase in this embodiment includes:

(1) Preparation method of polyethylene glycol modified uricase: According to the literature method, the uricase of Candida utilis was modified with polyethylene glycol. After modification, it was separated and purified to remove excess polyethylene glycol from the reaction to prepare enough polyethylene glycol modified uricase protein sample.

(2) Take 60% of the total weight of water for injection, add glycerin, propylene glycol, EDTA-2Na, and dissolve them and stir evenly; then add methyl cellulose, stir evenly after dissolution, sterilize with moist heat at 115° C. for 30 minutes for use Thing;

(3) Add benzoic acid, mannitol, and the main drug polyethylene glycol modified uricase respectively to an appropriate amount of water for injection to dissolve, filter and sterilize through a 0.22 μm microporous membrane for use;

(4) Add the benzoic acid of (3) to the spare material of (2), adjust the pH to 8.0 with 10% NaHCO₃ solution, and then add the mannitol of (3) and the main drug (polyethylene glycol modified uricase). Stir it evenly and dispense it into plastic tubes to obtain the finished product of uricase gel preparation for external use, and store it in a refrigerator at 4° C. for later use.

### Example 5. Preparation Scheme 5 of Uricase Gel Preparation for External Use

The gel preparation for external use of uricase in this embodiment is composed of the following components (calculated as 100 g gel):

Fc-uricase fusion protein (main drug) 50 mg (0.05%)

Carbomer 942 (aqueous gel base) 0.3 g (0.3%)

Mannitol (protein protective agent) 9 g (9%)

Absolute ethanol (humectant) 15 g (15%)

EDTA-2Na (chelating agent) 40 mg (0.04%)

Propylene glycol (dermal penetration enhancer) 2.5 g (2.5%)

Benzoic acid (preservative) 5 mg (0.005%)

The balance is water for injection.

Use 10% NaHCO₃ solution (pH adjuster) to adjust the pH of the formulation to 8.0.

The preparation method of the gel preparation for external use of uricase in this embodiment includes:

(1) Take 60% of the total water for injection, add propylene glycol, absolute ethanol, EDTA-2Na, and dissolve and stir evenly; then add carbomer 942, after night swelling and dissolution, sterilize with moist heat at 115° C. for 30 minutes Get a spare;

(2) Add benzoic acid, mannitol, and the main drug Fc-uricase fusion protein to an appropriate amount of water for injection to dissolve, and filter and sterilize through a 0.22 μm microporous membrane for use;

(3) Add the benzoic acid of (2) to the spare part of (1), adjust the pH to 8.0 with 10% NaHCO₃ solution, then add the mannitol of (2), the main drug Fc-uricase fusion protein and appropriate amount of water for injection, stir Evenly, dispense into plastic tubes to obtain the finished product of uricase gel preparation for external use, and store it in a refrigerator at 4° C. for later use.

### Example 6. Preparation Scheme 6 of Uricase Gel Preparation for External Use

The gel preparation for external use of uricase in this embodiment is composed of the following components (calculated as 100 g gel):

Recombinant human-pig chimeric uricase (main drug) 1 g (1.0%)

Gum Arabic (aqueous gel base) 20 g (20%)

Sucrose (protein protective agent) 15 g (15%)

Mannitol (protein protective agent) 5 g (5%)

Glycerin (humectant) 25 g (25%)

EDTA-2K (chelating agent) 0.2 g (0.2%)

Azone (transdermal enhancer) 3 g (3%)

Sodium benzoate (preservative) 0.5 g (0.5%)

The balance is water for injection.

Use 15% triethanolamine solution (pH adjuster) to adjust the pH of the formulation to 7.4.

The preparation method of the gel preparation for external use of uricase in this embodiment includes:

(1) Take 60% of the total weight of water for injection, add azone, glycerin, EDTA-2K, and dissolve and stir evenly; then add gum arabic, swell overnight, and sterilize with moist heat at 115° C. for 30 minutes to obtain a spare;

(2) Dissolve sodium benzoate, sucrose, mannitol, and the main drug recombinant human-pig chimeric uricase in an appropriate amount of water for injection, and filter and sterilize them through a 0.22 μm microporous membrane for use;

(3) Add the sodium benzoate of (2) to the spare material of (1), adjust the pH to 7.4 with 15% triethanolamine solution, and then add the sucrose of (2), mannitol, the main drug recombinant human-pig chimeric uricase and appropriate amount of water for injection, stir evenly, and dispense into sterile aluminum tubes to obtain the finished product of uricase gel preparation for external use, and store it in a refrigerator at 4° C. for later use.

### Example 7. Preparation Scheme 7 of Uricase Gel Preparation for External Use

The gel preparation for external use of uricase in this embodiment is composed of the following components (calculated as 100 g gel):

Candida utilis uricase (main drug) 0.5 g (0.5%)

Methyl cellulose (aqueous gel matrix) 6 g (6%)

Mannitol (protein protective agent) 10 g (10%)

Glycerin (humectant) 15 g (15%)

EDTA-2Na (chelating agent) 0.1 g (0.1%)

Propylene glycol (dermal penetration enhancer) 1.5 (1.5%)

Sodium benzoate (preservative) 0.25 g (0.25%)

The balance is water for injection.

Use 15% NaOH solution (pH adjuster) to adjust the pH of the formulation to 7.5.

The preparation method of the gel preparation for external use of uricase in this embodiment includes:

(1) Take 60% by weight of water for injection, add propylene glycol, glycerin, sodium benzoate, and dissolve and stir evenly; then add methyl cellulose, after night swelling and dissolution, sterilize with moist heat at 115° C. for 30 minutes to obtain a spare;

(2) Add EDTA-2Na, mannitol, and the main drug Candida utilis uricase respectively to an appropriate amount of water for injection to dissolve, filter and sterilize through a 0.22 μm microporous membrane for use;

(3) Add the sodium benzoate of (2) to the spare of (1), adjust the pH to 7.5 with 15% NaOH solution, and then add the mannitol of (2), the main drug Candida utilis uricase, and an appropriate amount of water for injection, stir uniformly, and dispense into sterile polyethylene composite hoses to obtain the finished product of uricase gel preparation for external use, and store it in a refrigerator at 4° C. for later use.

### Example 8. Preparation Scheme 8 of Uricase Gel Preparation for External Use

The uricase gel preparation for external use of this embodiment consists of the following components (calculated as 100 g gel):

Uricase from Aspergillus flavus (main drug) 0.35 g (0.35%)

Carbomer 934 (aqueous gel base) 0.05 g (0.05%)

BSA (protein protective agent) 0.15 g (0.15%)

Glycerin (humectant) 2 g (2%)

EDTA-2K (chelating agent) 10 mg (0.01%)

Hyaluronic acid (transdermal enhancer) 5 mg (0.005%)

Methyl p-hydroxybenzoate (preservative) 10 mg (0.01%)

The balance is water for injection.

Use 15% NaOH solution (pH adjuster) to adjust the pH of the formulation to 8.5.

The preparation method of the gel preparation for external use of uricase in this embodiment includes:

(1) Take 60% of the total weight of water for injection, add glycerin, hyaluronic acid, EDTA-2K and dissolve and stir evenly; then add carbomer 934, after night swelling and dissolution, sterilize with moist heat at 115° C. for 30 minutes Get a spare;

(2) Methylparaben, BSA, and the main drug Aspergillus flavus-derived uricase were added to the appropriate amount of water for injection to dissolve, and then filtered through a 0.22 μm microporous membrane to sterilize for use;

(3) Add the methyl parahydroxybenzoate of (2) to the spare of (1), adjust the pH to 8.5 with 15% NaOH solution, then add the BSA of (2), the main drug Aspergillus flavus-derived uricase, and an appropriate amount of injection stir evenly with water, and dispense it into medical aluminum-plastic tubes to obtain the finished product of uricase gel preparation for external use, and store it in a refrigerator at 4° C. for later use.

### Example 9. Study on the Stability of Uricase Gel Preparation for External Use and the Stability of Enzyme Activity

A. Study on the Stability of Uricase Gel Preparations for External Use

Three batches of samples were prepared according to the preparation method of the gel preparation for external use of uricase in Example 1, and the production batch numbers were 20170912, 20170913, and 20170914 respectively. The stability of the gel preparation was studied by the following method.

Place the above-mentioned gel preparation uricase external use gel preparation in a closed sterile aluminum tube and store it at 37° C., room temperature and 4° C. respectively, and regularly check the appearance, dehydration, pH and mildew of the gel preparation. And take samples to determine the retention rate of uricase enzyme activity in the gel preparation, and test the stability of uricase enzyme activity in the gel preparation uricase external gel preparation.

The results showed that after three batches of gel preparations uricase external use gel preparations were stored at 37° C. for 3 months and at room temperature and 4° C. for 6 months, the gel preparations had no water loss, no mildew, and no changes in appearance and pH. (Note: Due to space limitations, specific experimental data are not listed here)

B. Determination of Standard Curve for Determination of Uricase Activity

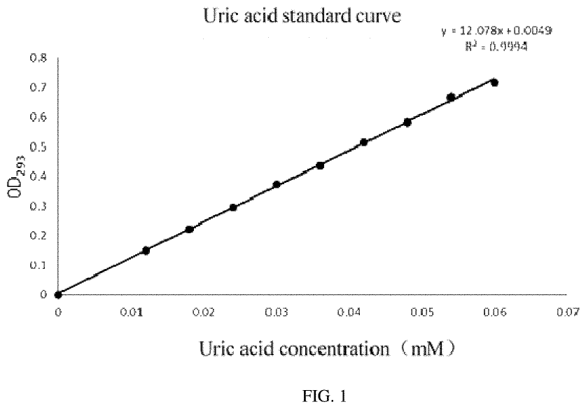

Determination of uric acid standard curve: use 0.1M borax-boric acid solution (pH8.5) to dilute the prepared 60004 uric acid mother solution to 60 μM, 5404, 4804, 4204, 3604, 30 μM, 24 μM, 18 μM, 12 μM, 0 μM, and measure each the OD₂₉₃ results of the solution are shown in Table 1: According to the measured data, a standard curve is drawn, and the results are shown in FIG. 1.

TABLE 1
Preparation of uric acid gradient concentration solution

| Number | 1 | 2 | 3 | 4 | 5 | 6 | 7 | 8 | 9 | 10 |
|---|---|---|---|---|---|---|---|---|---|---|
| 600 μM uric acid mother | 0 | 0.08 | 0.12 | 0.16 | 0.2 | 0.24 | 0.28 | 0.32 | 0.36 | 0.4 |
| liquor/mL | | | | | | | | | | |
| 0.1M borax-boric acid | 4.0 | 3.92 | 3.88 | 3.84 | 3.80 | 3.76 | 3.72 | 3.68 | 3.64 | 3.6 |
| solution (pH 8.5)/mL | | | | | | | | | | |
| Uric acid concentration/mM | 0 | 0.012 | 0.018 | 0.024 | 0.030 | 0.036 | 0.042 | 0.048 | 0.054 | 0.060 |
| OD₂₉₃ | 0 | 0.150 | 0.223 | 0.296 | 0.373 | 0.438 | 0.514 | 0.583 | 0.667 | 0.718 |

C. Uricase activity detection:

According to the linear range of the measured uric acid standard curve, add uric acid solution to the cuvette, add an appropriate amount of enzyme solution to the uric acid solution according to the concentration of the enzyme solution to ensure that the substrate is excessive, mix the two quickly and measure the 293 nm within 3 minutes. According to the standard curve of uric acid, calculate the consumption of uric acid and calculate the enzyme activity. The formula is as follows, where U=Uricase activity unit; (Ao−A)/3 represents the slope of the decrease in absorbance of uric acid solution at 293 nm within 3 min of reaction; Vt=total volume of reaction solution (mL); 12.078 is uric acid at 293 nm Micromolar extinction coefficient at the position: Ve=Enzyme liquid volume (mL).

$$U = \frac{( {A}_{0} - A ) / 3 \times {V}_{t}}{12.078 \times Ve} U = \frac{( {A}_{0} - A ) / 5 \times {V}_{t}}{12.078 \times Ve}$$

D. Study on the Enzyme Activity Stability of Human-Pig Chimeric Gel Preparation Uricase Gel Preparation for External Use

Place the recombinant human-pig chimeric gel preparation uricase gel preparation for external use under the conditions of 4° C. and 37° C., and sample at different time points to detect the recombinant human-pig chimeric uricase activity. The results are shown in the following table:

TABLE 2
Enzyme activity stability of 37° C. gel preparation

| Time/h | 0 | 2 | 4 | 8 | 12 | 24 | 48 | 72 |
|---|---|---|---|---|---|---|---|---|
| Enzyme activity | 100 | 97 | 97 | 97 | 93 | 92 | 85 | 81 |
| retention rate % | | | | | | | | |

It can be seen from Table 2 that the enzymatic activity retention rate of the gel preparation uricase external use gel preparation at 37° C. for 24 hours remains above 90%, and the enzymatic activity retention rate can still reach 85% at 48 hours and 81%, 72 hours.

TABLE 3
Enzyme activity stability of 4° C. gel preparation
uricase gel preparation for external use

| Time/week | 0 | 1 | 3 | 7 | 11 | 20 | 24 |
|---|---|---|---|---|---|---|---|
| Enzyme activity | 100 | 99 | 93 | 91 | 93 | 86 | 86 |
| retention rate % | | | | | | | |

It can be seen from Table 3 that the retention rate of the enzyme activity of the gel preparation uricase external use gel preparation at 4° C. for 6 months remains above 85%.

In summary, the uricase gel preparation for external use of the present invention can protect uricase from the influence of various external factors, is beneficial to stabilize uricase and protect the activity of uricase.

### Example 10 Local Irritation Test of Uricase Gel Preparation for External Use

Take 6 healthy rabbits, weighing 1.8-2.2 kg, shave symmetrically on both sides of the back spine, about 4×4 cm² on each side, rest for 24 hours, and raise them normally. Apply an appropriate amount of the urinase external gel preparation of Example 1 on one side, and apply an appropriate amount of the aqueous gel matrix used in Example 1 on the other side. The residues were washed off for 24 hours. Observed continuously for 20 days, the rabbit skin did not appear erythema, papules, phenomenon such as blisters.

### Example 11. Determination of Skin Uric Acid Content

A. Uric Acid Standard Curve Drawing

(1) Method for Detecting Uric Acid

Chromatographic conditions: Waters C18 column (250 mm×4.6 mm, 5μm); Mobile phase: 3 mL phosphoric acid, 30 mL methanol, add ultrapure water to constant volume; Flow rate: 1.0 mL/min; Detection wavelength: 293 nm;

Column temperature: 30° C.; injection The volume is 20 μL.

(2) Preparation of Uric Acid Mother Liquor (5000 μM):

Weigh 84 mg of uric acid in a 100 mL beaker, add an appropriate amount of borax-boric acid buffer solution, and stir magnetically. Stir it with a stirrer until it is completely dissolved, and dilute to 100 mL to get it.

(3) 0-500 μM Uric Acid Standard Curve Drawing:

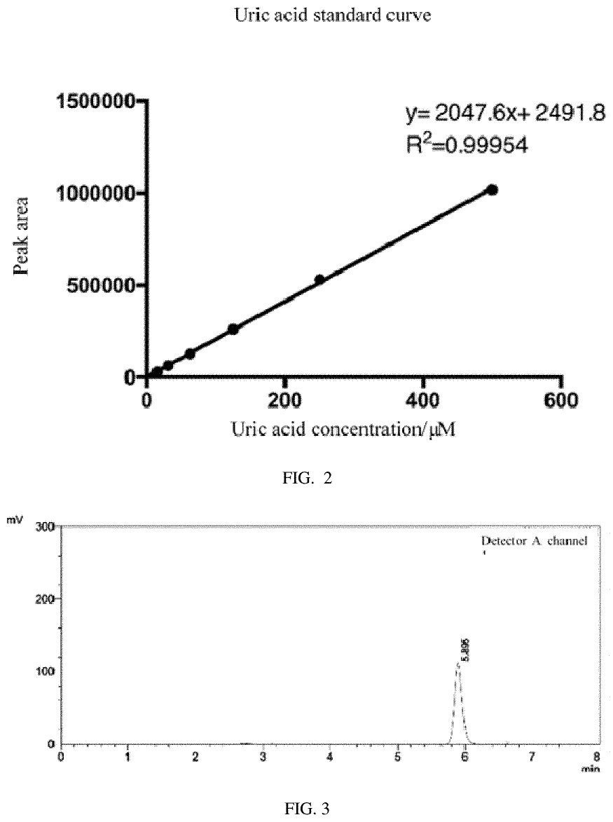

Take 5 mM uric acid, dilute to 500, 250, 100, 50, 20, 10 μM with borax-boric acid buffer solution successively, draw 20 μL of the above solution, add protein precipitation agent at a volume ratio of 1:10, ice-water bath for 5 minutes, centrifuge and pipette up after clearing the sample, draw a standard curve based on the uric acid concentration and peak area, as shown in FIG. 2: An example of the chromatogram for the detection of uric acid solution is shown in FIG. 3.

B. HPLC Determination of Uric Acid Content on the Skin Surface

Twenty-four male Kunming mice aged 5-6 weeks were randomly divided into the following 4 groups according to body weight: (1) control group, (2) model group, (3) negative control group, (4) medication of uricase gel group. There are 6 mice in each group. The mice in the control group have a normal diet and are not given uricase topical gel preparation or water-based gel matrix; mice in the model group are given daily intraperitoneal injection of sodium urate suspension and mixed with yeast powder feed to prepare a mouse hyperuricemia model; the negative control group's modeling method is the same as that of the model group. After the modeling is successful, give the aqueous gel matrix (blank gel) used in Example 1; uricase gel medication group: The modeling method is the same as that of the model group. After the modeling is successful, the uricase external gel preparation of Example 1 is administered.

Before the first medication, the exposed skin of the back of each group of mice was disinfected and cleaned with a cotton ball. After wiping clean, the mice were put back into the cage. The gel medication group was given uricase topical gel, and the negative control group was given blank gel. After the gel is naturally air-dried, the mice are put back into the cage, free to eat and drink, and at different times, use a cotton swab to dip an appropriate amount of normal saline to wipe the back skin of each group of mice, and then squeeze the water on the cotton swab into the EP tube, Labeled as “skin wipe sample”. After centrifugation, a certain proportion of protein precipitant is added to the obtained sample, and the supernatant is taken for HPLC determination after centrifugation. The peak area of uric acid is obtained by the integration method, and then the uric acid concentration is calculated according to the standard curve.

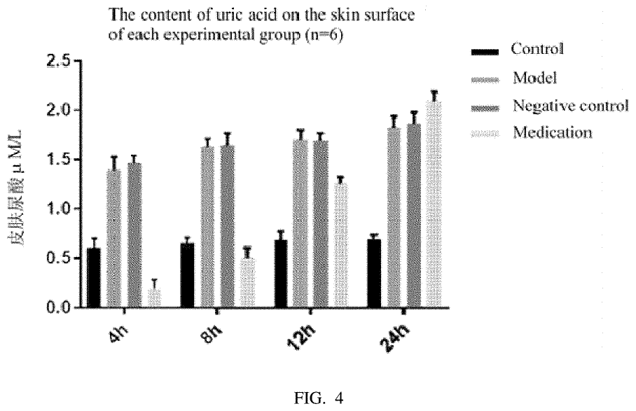

It can be seen from FIG. 4 that the uric acid concentration on the skin surface of the control group and the model group gradually reached the upper limit after 8 hours, and the uric acid content of the skin surface of the model group was 2.4 times that of the control group. In the medication group, because the uricase in the gel can continuously degrade uric acid secreted from sweat glands to the skin surface, the uric acid content on the skin surface is lower than that of the control group after 4-8 hours, but exceeds that of the control group after 12 hours. After 24 hours, the concentration of uric acid on the skin surface exceeding the model group. Because the skin surface uric acid and serum uric acid concentration are proportional to each other, due to the decrease of the skin surface uric acid concentration, a concentration gradient of uric acid inside and outside the skin is formed, so that uric acid in the body is continuously discharged to the skin surface through sweat glands. The excretion of uric acid on the skin surface of the uricase gel medication group significantly exceeded that of the model group. Therefore, it can be shown that the uricase gel can not only degrade uric acid on the skin surface, but also promote skin uric acid excretion.

C. Determination of Uric Acid Content in Skin Tissue by High Performance Liquid Chromatography

One week after the medication, the mice were sacrificed by cervical dislocation. The skin tissues of each group of mice were removed and homogenized. After centrifugation, the supernatant was transferred to an EP tube, and a certain proportion of protein precipitation agent was added. After centrifugation, the supernatant was used for HPLC. Measure and integrate the method to obtain the peak area of uric acid, then calculate the uric acid concentration according to the standard curve, and then obtain the skin uric acid content per unit weight according to the following formula:

Unit skin uric acid content=skin uric acid content (μM)/skin weight (g)

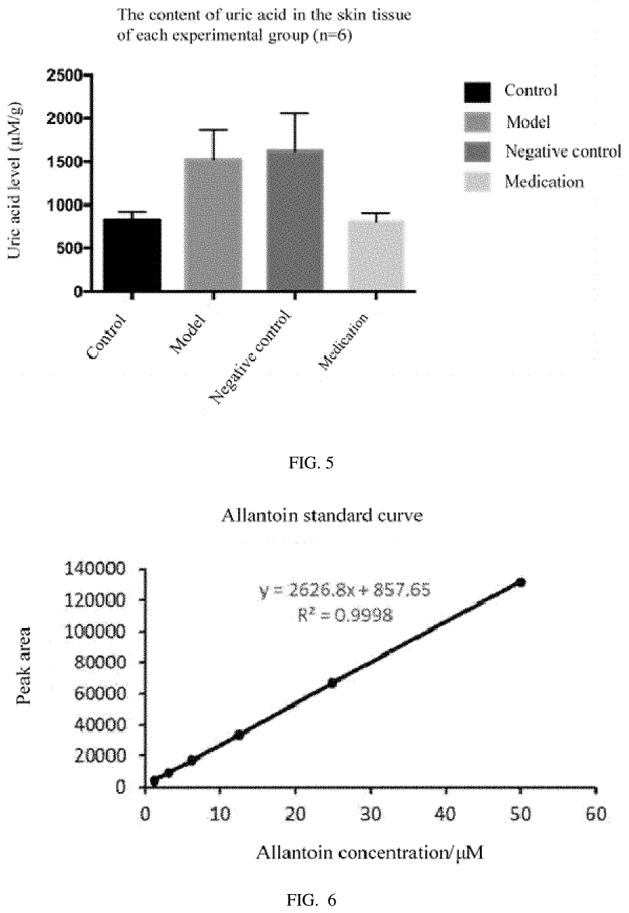

It can be seen from FIG. 5 that the skin tissue uric acid level of hyperuricemia mice (model group) is twice that of normal mice (control group). The negative control group was given a blank gel and did not have the effect of breaking down uric acid, so it could not prevent the increase in skin uric acid level of mice. The uric acid level of skin tissue was close to that of the model group, and it was still twice the skin uric acid level of the control group. The medication group was given a topical gel preparation of uricase. Because uricase can degrade uric acid, it can degrade the uric acid that is continuously secreted out of the skin, thereby reducing the level of uric acid in the skin tissue. And one week after the medication, the skin tissue uric acid levels of the mice in the medication group and the control group were similar, indicating that the medication of a week-long uricase topical gel preparation can significantly reduce the uric acid in the skin tissue of the mice. The blood uric acid level of mice is directly proportional to the skin uric acid level. The decrease of the uric acid level of the skin tissue means that the blood uric acid level of the mice will also decrease. Uricase topical gel preparations play a role in reducing blood uric acid levels in this way.

### Example 12, Detection of Skin Allantoin Content

A. Drawing the Standard Curve of Allantoin

(1) Method of Detecting Allantoin

Chromatographic conditions: Dubhe: C18 column (250 mm×4.6 mm, 5 μm); mobile phase: methanol-water (10:90); column temperature: 30° C.;

detection wavelength: 210 nm; flow rate: 1 mL×min⁻¹; injection 10 μL

(2) Preparation of Allantoin Mother Liquor (500 μM):

Use an analytical balance to accurately weigh 0.02 g allantoin standard in 100 mL water, stir and dissolve it fully, drain into a 250 mL clean volumetric flask with a glass rod, continue to add water to make the volume up to the mark, and mix well to obtain a 500 μM allantoic sac Vegetarian standard solution.

(3) 0-500 μM Allantoin Standard Curve Drawing:

Take 500 μM allantoin solution, dilute with distilled water to 250, 100, 50, 20, 10 μM in turn, draw 204, of the above solution, add protein precipitation agent in a certain proportion, bath in ice water for 5 minutes, centrifuge, draw the supernatant and inject the sample with allantoic draw a standard curve for the element concentration and peak area, as shown in FIG. 6.

B. HPLC Determination of Allantoin Content on the Skin Surface after Medication

Take the collected “skin wipe samples” of the uricase gel medication group, centrifuge to take the supernatant and add a certain proportion of protein precipitant. After centrifugation, the supernatant is used for HPLC detection. The allantoin peak area is obtained by integration method, and then according to the standard the curve calculates the concentration of allantoin.

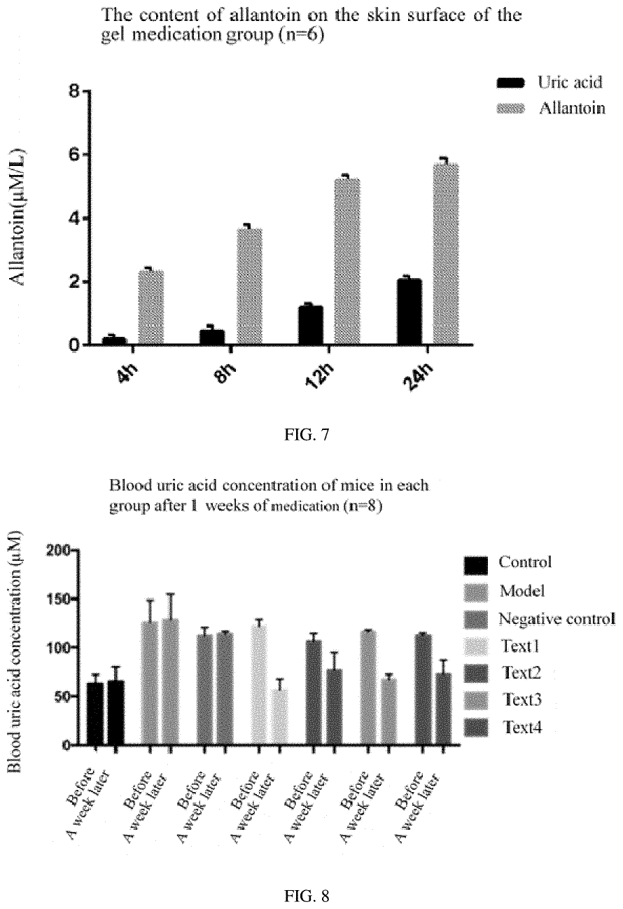

The results are shown in FIG. 7. After 4 hours of medication, the uric acid gel on the skin surface of the medication group can continuously degrade the uric acid excreted from the body to the skin surface to produce allantoin, so the uric acid content on the skin surface is lower than control group. 4-12 h, the production of allantoin continues to rise, indicating that the gel still retains enzyme activity and has the ability to degrade skin uric acid. Up to 24 h, the amount of allantoin produced at this time was 0.3 μM more than that of 12 h, and the two were similar. It is speculated that the uricase in the gel has been fully reacted at this time, or it is degraded by the protease on the skin surface and the mouse Inactivated by licking. But at this time, uric acid is still continuously excreted from the body to the body, and continuously accumulates on the surface of the skin. In the end, the uric acid excretion of the drug group for 24 hours exceeds the uric acid content of the skin surface of the model group for 24 hours. Therefore, it can be explained that the uricase gel preparation can convert the uric acid on the skin surface into the more water-soluble allantoin, and after 12 hours of medication, the uricase gel preparation on the skin surface still remains active. The blood uric acid is continuously excreted, and the excretion of skin uric acid increases, which plays a role in rapidly and continuously lowering the blood uric acid level.

### Example 13. Pharmacodynamic Study of Uricase Gel Preparation for External Use

A: Modeling Mice with Hyperuricemia

The control group was fed with ordinary feed only; the model group, the negative control group, and the medication group were intraperitoneally injected with sodium urate suspension once a day, and the yeast powder-containing feed was given by the mixing method.

B: Animal Grouping and Medication

Fifty-six male Kunming mice aged 5-6 weeks were randomly divided into the following 7 groups according to body weight: control group, model group, negative control group, medication group 1 (UOX₈₃ gel: the recombinant human-pig chimeric uricase gel preparation of Example 1 for external use, specification 25 mg/100 g, batch; 20180512), medication group 2 (UOX_{PEU} gel: Candida utilis uricase external use gel preparation of Example 2, specification 25 mg/100 g, batch: 20180512), medication group 3 (polyethylene glycol modified uricase external use gel preparation of Example 4, Specification 25 mg/100 g, batch: 20180513), medication group 4 (the Fc-uricase fusion protein topical gel preparation of Example 5, specification 25 mg/100 g, batch: 20180513).

Control group: no modeling and no medication; model group: daily modeling and no medication; negative control group: continuous modeling, back hair removal and applying blank gel (using the aqueous gel matrix of Example 1); medication Group: Continuous modeling. After successful modeling, the back hair was removed and the corresponding uricase topical gel preparation was applied daily; except for the control group without modeling, other mice were normally modeled during the medication period.

C. Determination of Blood Uric Acid Level

After the continuous medication for one week, blood was taken from the orbit of each group of mice. Let it stand for a period of time at 4° C., centrifuge to take the supernatant, inject 20 of HPLC sample, record the uric acid peak area, and calculate the blood uric acid content according to the uric acid standard curve.

TABLE 4
Serum uric acid levels of mice in each group after one week of continuous
medication (x̅ ± s, n = 8, μM)

| Project | Control | Model | Negative control |
|---|---|---|---|
| Before medication | 63.243 ± 9.232 | 125.457 ± 23.132 | 112.317 ± 8.518 |
| After medication | 65.527 ± 14.647 | 128.323 ± 26.222 | 114.067 ± 2.829 |

| Project | Text1 | Text2 | Text3 | Text4 |
|---|---|---|---|---|
| Before | 121.937 ± 6.672 | 106.433 ± 8.377 | 116.220 ± 2.224 | 112.433 ± 2.829 |
| medication | | | | |
| After | 56.673 ± 11.632*** | 76.530 ± 18.919* | 67.643 ± 4.942** | 72.393 ± 15.071** |
| medication | | | | |

Note:
*, * * and * * *respectively indicate the significance of the difference between the model group and the model group at the level of P < 0.05, P < 0.01, P < 0.001.

It can be seen from the above table and FIG. 8 that the serum uric acid levels of the mice in the model group, the negative control group, the medication group 1, the medication group 2, the medication group 3, and the medication group 4 after the hyperuricemia model are as follows: The serum uric acid level of mice in the control group was more than twice as high. One week after the medication, the blood uric acid levels of the mice in the medication group 1, the medication group 2, the medication group 3 and the medication group 4 decreased to similar to the control group.

In summary, the uricase gel preparation for external use of the present invention not only significantly degrades the uric acid on the skin surface to be converted into allantoin, but also promotes the continuous excretion of blood uric acid to the skin surface, thereby reducing the serum uric acid level.

In addition to the above-mentioned embodiments, the present invention can also have other embodiments. All technical solutions formed by equivalent replacements or equivalent transformations fall within the protection scope of the present invention.

What is claimed is:

1. A gel preparation for external use of uricase, characterized in that: the preparation comprises the following components by weight:
greater than 0 and less than or equal to 1.0% of uricase,
0.05-20% of aqueous gel matrix,
0.15-20% of protein protective agent,
2-25% of humectant,
0.01-0.20% of chelating agent,
0.001-3.0% of penetration enhancer,
0.005-0.5% of preservative and
water for injection making the rest of the preparation;
pH of the preparation is adjusted to 5-10 by a pH adjuster.

2. The gel preparation for external use of uricase according to claim 1, wherein the uricase is natural uricase, recombinant uricase, chimeric uricase or fusion uricase; wherein the natural uricase is made from prokaryotes or eukaryotes, the recombinant uricase, chimeric uricase, and fusion uricase are prepared by bioengineering technology; wherein the prokaryotes and eukaryotes are microorganisms and mammals, the microorganisms are Candida utilis and Aspergillus flavus; the mammals are pig and dog; the recombinant uricase is expressed by a natural uricase gene; the chimeric uricase is an uricase chimeric protein recombinantly expressed by a nucleotide sequence consisted of different natural uricase chimeric genes; the fusion uricase is a recombinantly expressed human serum albumin-uricase fusion protein genes (fusion of the natural uricase gene and a human serum albumin gene), or recombinantly expressed Fc-uricase fusion protein (fusion of the natural uricase gene and a humanized antibody Fc fragment nucleotide sequence).

3. The gel preparation for external use of uricase according to claim 1, wherein the uricase is modified chemically which includes PEG modification.

4. The gel preparation for external use of uricase according to claim 1, wherein the aqueous gel matrix is selected from the group consisting of natural polymer, semi-synthetic polymer or synthetic polymer; wherein the natural polymer is starch, alginate, gum arabic, tragacanth, agar, or gelatin; the semi-synthetic polymer is a modified cellulose and a modified starch, the modified cellulose is a carboxymethyl cellulose and a methyl cellulose element; the synthetic polymer is carbomer and sodium polyacrylate.

5. The gel preparation for external use of uricase according to claim 4, wherein the aqueous gel matrix is one or more carbomer selected from the group consisting of carbomer 934, carbomer 940, carbomer 941, carbomer 942, carbomer 971, carbomer 974, carbomer 980 and carbomer 981.

6. The gel preparation for external use of uricase according to claim 1, characterized in that the protein protective agent is one or more compound(s) selected from the group consisting of bovine serum albumin, mannitol, sucrose, sodium citrate and sorbitol; wherein
the humectant is one or more compound(s) selected from the group consisting of glycerin, propylene glycol, ethanol and hyaluronic acid;
the chelating agent is one or more salt(s) selected from the group consisting of salt of ethylenediaminetetraacetic acid and salt of ethylenediaminetetraacetic acid class that is ethylenediaminetetraacetic acid disodium salt or ethylenediaminetetraacetic acid dipotassium salt;
the penetration enhancer is one or more compound(s) selected from the group consisting of azone, propylene glycol, hyaluronic acid, cholate, deoxycholate, urea, cyclodextrin and Tween-80;
the preservative is one or more compound(s) selected from the group consisting of benzoic acid, sodium benzoate, methyl p-hydroxybenzoate, ethyl p-hydroxybenzoate, phenol and sorbic acid;
the pH adjusting agent is one or more compound(s) selected from the group consisting of triethanolamine, NaOH and NaHCO₃.

7. The gel preparation for external use of uricase according to claim 1, wherein content of the uricase is 0.001-0.1% by weight, the pH of the preparation is 6-9.

8. A method for preparing the uricase gel preparation for external use according to claim 1, comprising the following steps:
i) take an appropriate amount of the water for injection, add the penetration enhancer, moisturizer, and the chelating agent, and dissolve and stir evenly, then add the aqueous gel matrix, swell and/or dissolve and then stir evenly, and sterilize with moist heat;
ii) dissolve the protein protectant, the preservative and the uricase in the water for injection, respectively; and filter and sterilize the protein protectant, the preservative and the uricase through a microporous membrane, respectively;
iii) mix solution obtained in the step i) with the preservative obtained in the step ii), and adjust the pH value to 5-10 with the pH regulator, then, add the protein protectant obtained in the step ii), the uricase and the water for injection which yields the uricase gel preparation, the uricase gel preparation is uniformly stirred and packaged.

9. The method according to claim 8, characterized in that, in the step i), time for swelling is overnight, and condition of moist heat sterilization is 115° C. for 30 minutes; in the step ii), pore size of the microporous film is 0.22 μm; in the step iii), the pH is 6-9, the uricase gel preparation packed into pharmaceutically acceptable containers which comprise pharmaceutical aluminum tubes, plastic tubes, aluminum-plastic tubes, and polyethylene composite tubes; or coat the uricase gel preparation on a sanitary material to make a gel patch; store the uricase gel preparation in a refrigerator.

10. A method for treating a disease by decreasing serum uric acid comprising a step of administrating a subject in need with the gel preparation of claim 1, wherein the disease is hyperuricemia, gout or diseases related to the hyperuricemia.
